(19) United States
(12) Patent Application Publication
(10) Pub. No.: US 2021/0220422 A1
(43) Pub. Date: Jul. 22, 2021

(54) DIETARY SUPPLEMENT COMPOSITIONS AND METHODS

(71) Applicant: Jonathan Parker, West Palm Beach, FL (US)

(72) Inventor: Jonathan Parker, West Palm Beach, FL (US)

(21) Appl. No.: 16/747,312

(22) Filed: Jan. 20, 2020

(51) Int. Cl. A61K 36/81; A61K 36/41; A61K 36/79; A61K 36/28; A61K 36/258; A61K 35/02; A61K 36/25; A61K 36/9068; A61K 36/484; A61K 36/67; A61K 31/455; A61K 33/00; A61K 31/375; A61K 31/593; A61K 33/06; A61K 36/48; A61K 36/185; A61K 31/404; A61K 33/22; A61K 33/30; A61K 33/24; A61K 33/18; A61K 33/04; A61K 36/53; A61K 31/714; A61K 36/73; A23L 33/105; A23L 33/155; A23L 33/16

(52) U.S. Cl. CPC A61K 36/81; A23V 2002/00; A61K 36/79; A61K 36/28; A61K 36/258; A61K 35/02; A61K 36/25; A61K 36/9068; A61K 36/484; A61K 36/67; A61K 31/455; A61K 33/00; A61K 31/375; A61K 31/593; A61K 33/06; A61K 36/48; A61K 36/185; A61K 31/404; A61K 33/22; A61K 33/30; A61K 33/24; A61K 33/18; A61K 33/04; A61K 36/53; A61K 31/714; A61K 36/73; A23L 33/105; A23L 33/155; A23L 33/16; A61K 36/41

(57) ABSTRACT

Described are natural herbal supplement compositions that provide a sense of energy, stress relief, well-being, and vitality in humans and most mammals. In one aspect, the compositions include a unique combination of ingredients categorized into one of three groups, Adaptogens, Bioenhancers, and Circulation Enhancers, and selecting several plants from each group to create a base formula. The base formula is then combined with other supportive nutrients to form a nutrient supplement.

### REFERENCE TO RELATED APPLICATION

This application claims priority to U.S. Provisional Application No. 62/919,409, filed on Mar. 11, 2019. This application is hereby incorporated by reference in its entirety for all of its teachings.

### BACKGROUND

The study and use of medicinal herbs and other natural compounds for the treatment of diverse diseases date back over 5000 years and has been equal to or more pervasive than the study of conventional allopathic Western medicine. The active principles from many traditional medicines have been extracted from plants, the curative constituents identified and their mechanisms of action determined. Plant-based medicines are typically well-tolerated, with less severe side effects, and a wider range of benefits. However, despite the powerful medicinal actions of many plants, they are often individually insufficient to take chronic degenerative diseases into remission. In contrast, while synthetic drugs can be highly effective, their use is often hampered by severe side effects and a very narrow range of benefits. Accordingly, there is a need for improved treatment regimens that provide sufficient efficacy with improved patient tolerance. There is also a need for nutraceutical and phytoceutical dietary supplements that support a healthy stress response in humans and other animals, giving the ability to combat and improve various diseases and health conditions. Further, there is a need for nutraceutical and phytoceutical dietary supplement formulations that are particular combinations of vitamins, minerals, herbs, and other nutrients and have a synergistic effect in combination.

### SUMMARY

An exemplary embodiment relates to a nutritional supplement. The supplement includes a base formula being formed from at least three categories of ingredients, the three categories being adaptogens, bioenhancers, and circulation enhancers and a nutrient formula, the nutrient formula being formed of at least one ingredient having an intended targeted effect on a user. The adaptogens are combined from at least three adaptogenic ingredients. The bioenhancers are combined from at least three bioenhancer ingredients and the circulation enhancers are combined from at least three circulation enhancing ingredients.

Another exemplary embodiment relates to a nutraceutical product. The product includes at least three adaptogenic ingredients each potentially having adaptogenic effects, at least three bioenhancer ingredients each potentially having bioenhancer effects, and at least three circulation enhancing ingredients each potentially having circulation enhancing effects. At least one nutrient ingredient potentially has an intended targeted effect on a user. The adaptogenic ingredients, the bioenhancer ingredients, and the nutrient ingredients are combined in a form suitable for consumption by a human being.

Yet another exemplary embodiment relates to a method of manufacturing a nutraceutical product. The method includes combining into a mixture at least three adaptogenic ingredients each potentially having adaptogenic effects, combining into the mixture at least three bioenhancer ingredients each potentially having bioenhancer effects, combining into the mixture at least three circulation enhancing ingredients each potentially having circulation enhancing effects, and combining into the mixture at least one nutrient ingredient, the nutrient ingredient potentially having an intended targeted effect on a user. The method also includes forming the mixture suitable for consumption by a human being.

In addition to the foregoing, other system aspects are described in the claims, drawings, and text forming a part of the disclosure set forth herein. The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the disclosures set forth herein.

### BRIEF DESCRIPTION OF THE DRAWINGS

FIG. 1 is a block diagram representation of a process for formulating a nutritional supplement.

The use of the same symbols in different drawings typically indicates similar or identical items unless context dictates otherwise.

### DETAILED DESCRIPTION

Modern life exposes us to a combination of daily acute and long-term chronic stressors, including but not limited to chemical exposure stress, biological stress, radioactive stress, mental stress, and physical stress. As a human or animal ages, the organism has greater difficulty overcoming and recovering from these stressors. This, in turn, further accelerates the catabolic aging process leading to an eventual breakdown of all biological mechanisms thereby causing a lack of energy and vitality and eventually leading to a disease state.

In accordance with exemplary embodiments, herbal dietary supplements are supplements for supporting a healthy stress response. These herbal dietary supplements confer resilience by inducing positive physiological and psychological benefits, blunting stress reactivity, protecting against potentially adverse behavioral and metabolic consequences of stressful events, and possibly reducing the severity of some chronic diseases.

By reducing the negative effects of stressors on the body these herbal dietary supplements also typically improve energy, hormone levels, mood, body weight, visceral fat, circulation, libido, sexual performance, sleep, mental clarity, stamina, vitality, and other issues related to and arising from the aging process that are exacerbated by stressors in humans and animals.

Conventional herbal supplements may be inadequate to achieve desired results because they are often formulated with low doses of the active constituents, the wrong types or combinations of herbs and extracts, and/or they lack bioavailability. In accordance with exemplary embodiments, formulations with high concentrations of natural herbal phytochemicals are carefully combined in a unique scientifically validated nutraceutical and phytoceutical formula using therapeutic amounts of herbs and other nutrients that complement and boost the effects of each other for improved effectiveness.

Described herein are stimulant-free compositions that provide a sense of energy, vitality, and stress relief while helping to balance the entire neuroendocrine system which allows the body to achieve a state of homeostasis and allostasis. In one aspect, the base composition includes a combination of three groups (A, B, C) of unique blends of natural herbs or herbal extracts, vitamins, minerals, and other phytochemicals that work in a synergistic manner such that group A (Adaptogens) contains at least one species of Ashwagandha, at least one type of Shilajit, at least one species of Schisandra, at least one species of Rhodiola, and at least one species of Eleuthero and group B (Bioenhancers) includes at least one species of Ginger, at least one species of Licorice, and at least one species of Pepper and Group C (Circulation Enhancers) includes at least one type of Vitamin D, at least one type of Niacin, at least one type of Nitrite, and at least one type of Magnesium. Additional supportive nutraceuticals, phytoceuticals, herbs, herbal extracts, vitamins, minerals, sweeteners, and other nutrients may be added to the base composition (groups A, B, C) to improve taste and to treat specific health conditions as outlined below in the example formulations.

In accordance with an exemplary embodiment, a base formula with added vitamins, minerals, herbs, and other nutraceuticals is used to make unique powerful natural herbal formulations specifically designed to synergistically work together to nourish the entire neuroendocrine system including the HPAT axis and combat every type of stress the body encounters. These nutraceutical and phytoceutical blends use therapeutic levels of potent phytochemicals that have been shown through scientific studies to give beneficial outcomes to humans and animals.

To make the base formula a number of known beneficial plants were classified according to their capacity to enhance the three main elements that support general overall health: Adaptogens (A), Bioenhancers (B), and Circulation Enhancers (C). A synergistic effect is expected when all three categories of herbs (A, B, C) are added to a formulation, preferably using at least three or more plants from each category. Thus, various exemplary embodiments are made using nutraceuticals and phytoceuticals with specific targeted actions combined with the base formula according to these principles. Three examples of formulations prepared in this way are provided and additional formulations are being prepared and tested.

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting. Other features and advantages of the various embodiments will become apparent from the following non-limiting description of preferred embodiments.

In this specification and in the claims that follow, reference will be made to a number of terms that shall be defined to have the following meanings:

Use of the word “plants,” is defined as the entire plant or that portion with medicinal activity and can be in whole, ground, or an extract form. Also included are purified active ingredients and derivatives thereof. However, it is possible that the best efficacy of plants used herein is achieved with the use of the entire plant or its extracts, rather than with the use of isolated active ingredients. It must also be noted that, as used in the specification and the appended claims, the singular forms “a,” “an” and “the” include plural referents unless the context clearly dictates otherwise. Thus, for example, the reference to “an Eleuthero” includes mixtures of two or more such species, and the like.

Further, although plants are named here according to commonly used nomenclature, with improving taxonomy plants are often reclassified. Whenever a plant is referenced, it includes related species with similar active ingredients.

“Pharmaceutically acceptable excipients”, “flow agents”, and “carriers” are used herein according to art accepted meanings, and includes those ingredients needed to formulate a medicine for mammalian use, including the use of gelatin or cellulose capsules.

“Optional” or “optionally” means that the subsequently described event or circumstance can or cannot occur and that the description includes instances where the event or circumstance occurs and instances where it does not. For example, the phrase “optionally Rhaponticum extract” means that Rhaponticum extract may be optionally added to the composition.

The term “adaptogen” refers to a natural herb that is non-toxic and increases the body's resistance to stressors (including physical, mental, radiological, chemical, and biological agents), trauma, anxiety, and fatigue and promotes or restores normal physiological and psychological functioning and homeostasis. All adaptogens contain antioxidants, but antioxidants are not necessarily adaptogens. Plants classified as adaptogens are those that revitalize one's state of health by regulating the neuroendocrine system, activating stem cells, strengthening immunological systems, optimizing cellular processes, combating the effects of aging, reducing DNA damage, supporting a healthy inflammation response, increasing vital energy, reducing all types of stress, improving libido, improving healthy gene expression (epigenetics), improving insulin sensitivity, reducing body fat, increasing muscle mass, supporting the HPAT axis, improving sleep, boosting mental function, detoxifying on a cellular level, improving mitochondrial function, slowing catabolism while increasing anabolism, supporting bone and joint health, and supporting a healthy cardiovascular system thus controlling and improving the interactions between the numerous systems in the body.

The term “bioenhancer” refers to a natural plant, herb, or other substance that increases the bioavailability of other nutrients. Without a bioenhancer, much of the nutrients may not be absorbed and fully utilized by the body and could be excreted as waste. A bioenhancer or bio-potentiator allows one to take less and get increased benefits through better absorption and utilization.

The term “circulation enhancer” refers to a natural plant, herb, or other substance that increases blood flow and circulation (carrying oxygen and other nutrients) throughout the body by both dilating blood vessels and reducing blood viscosity thereby allowing your lungs, heart, muscles, and other systems to function optimally and efficiently. Blood circulation in our body is driven by the heart that provides a constant supply of blood to the body through the blood vessels. This process ensures the transportation of oxygen and nutrients to every cell in your body and helps avoid potential diseases by transporting white blood cells where they are needed by your immune system. Good circulation is vital to organ and cellular health and helps remove waste from the body created by cells, glands, and different organs.

“Synergistic” or “synergy” is used herein to mean that the effect is more than its additive property. In preferred embodiments, the synergy is at least 1.5, 2, 5, or 10 fold.

The term “base formula” refers to a combined formula including group A (Adaptogens), group B (Bioenhancers), and group C (Circulation Enhancers). The base formula will always be combined with a supportive nutrient formula to target specific health conditions.

The term “supportive nutrients” refers to specific ingredients combined in a formulation designed to help with a certain health condition. The supportive nutrient formulation will always be combined with the base formula in a synergistic way.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5 and are present in such ratio regardless of whether additional components are contained in the compound. A weight percent of a component, unless specifically stated to the contrary, is based on the total weight of the formulation or composition in which the component is included.

The compositions are comprised of a combination of more than one herb, and specific chemical extracts from herbs, which are formulated in a unique combination such that the individual components are combined for their individual utilities in the context of a specific health condition, as well as for their synergistic effect in the complete composition. Each herb may be identified by its botanical characteristics, as well as by the chemical compounds contained within the plant that may be extracted by chemical manufacturing processes. Accordingly, chemical compounds and individual constituents of the herbs, individually and collectively, that mimic the effect of the herbs and herbal extracts may be substituted for the actual herbs obtained from nature without departing from the scope of exemplary embodiments described.

In use, the compositions of the invention can be administered orally in a powder, gel, liquid, food bar, candy, sublingual tablet, or capsule form and may be combined with food or a drink as part of a treatment regimen or as a dietary or fitness supplement. Once mixed with food or a drink, the composition provides additional antioxidants and nutrients to further supplement the food or drink it is mixed with. When provided to non-humans, the compositions of the invention may be administered separately or may be combined with ordinary feed or liquid nourishment to affect the alterations in disease conditions as described herein. For optimal results, the capsule form of this composition may be consumed two to four times a day. In some aspects, the composition can be added to fruit and other food groups.

Thus, exemplary compositions include the herbal compositions described herein in oral dosage form, or in combination with any of the usual pharmaceutical or nutritional media employed in the art for oral liquid preparations, e.g., suspensions, elixirs, and solutions. Generally, media containing water, oils, alcohols, flavoring agents, preservatives, coloring agents and the like may also be used for flavor, texture, or shelf-life enhancement. Carriers such as starch, glycerol, alcohol, sugars, diluents, granulating agents, flow agents, lubricants, binders, disintegrating agents, and the like may be used to prepare oral solids (e.g., a solid cake, powders, capsules, pills, and sublingual tablets). Controlled release forms may also be used. Because of their ease in administration, tablets, pills, and capsules represent advantageous oral dosage unit forms, in which case solid pharmaceutical carriers are employed. If desired, tablets may be sugar-coated or enteric-coated by standard techniques. While the herbs and herb extracts used are naturally derived from plants that are edible, the compositions of exemplary embodiments can be administered as herbs or direct extracts of herbs, but one or more herbs can also be substituted with the chemical compounds or functional equivalents that are derived from such herbs. For example, one component of an exemplary embodiment is Mucuna pruriens. The main functional ingredient of Mucuna pruriens is known to be L-dopa, an organic molecule whose structure and function are well known. Thus, consistent with exemplary embodiments, the Mucuna pruriens herb may be replaced in some applications with the organic molecule contained therein or a synthetic version. Moreover, the compositions of exemplary embodiments can be combined with ordinary foods to enhance the value of their health benefits. For example, the compositions can be mixed with water or an aqueous beverage including tea, milk, various soft drinks, coffee, smoothies, other supplemental beverages, food supplements, candy, or food bars, and virtually any other food that can be supplemented with powder, liquid or gel.

In one aspect, about 2 to 6, or 3 grams of the powder can be added to 8-10 oz of a beverage or gel such as for example, tea, water, milk, coffee, various soft drinks, smoothies, and other supplemental beverages. The compositions may be consumed as many times as needed, with one to four times a day being optimal.

The advantages including convenience and uniqueness of exemplary embodiments will be set forth in part in the description which follows, and in part will be obvious from the description, or may be learned by practice of the aspects described below. Further, because the herbal dietary supplement may be embodied in the form of a capsule, sublingual tablet, powder, liquid beverage, gel, or food bar, the herbal dietary supplement is conveniently stored and transported, thereby permitting the herbal dietary supplement to be consumed at home, at work, in a vehicle, at the gym, or at other locations. The advantages described below will be realized and attained by means of the elements and combinations particularly pointed out in the appended claims. It should be understood and appreciated that in other example embodiments, both the foregoing general description and the following detailed description are exemplary and explanatory only and are not restrictive. All of the herbal dietary supplements may be prepared in other forms and may include the same or other ingredients in various amounts along with the base composition that includes groups A, B, and C.

### Example Base Formula:

Group A: An exemplary embodiment of group A (Adaptogens) provides compositions of an herbal supplement in the form of a powder in capsules comprising extracts of at least five (and sometimes up to forty one) ingredients selected from the group comprising at least one species of Eleuthero, at least one species of Ajuga turkestanica, at least one type of Shilajit, at least one type of Fulvic Acid, at least one type of Mumie, at least one species of Rhodiola, at least one species of Ginseng, at least one species of Rhaponticum, at least one type of Ecdysteroid, at least one species of Ashwagandha, at least one species of Holy Basil, at least one species of Aralia, at least one species of Prince Seng, at least one species of Dong Quai, at least one species of Maca, at least one species of Schisandra, at least one type of Cannabidiol, at least one species of He Shou Wu, at least one type of Turkesterone, at least one species of Amla, at least one species of Sorbus aucuparia, at least one species of Viburnum sargentii, at least one species of Rosa majalis, at least one type of Alpha-Casozepine, at least one type of Ecdysterone, at least one species of Jiaogulan, at least one species of Gynostemma pentaphyllum, at least one species of Cyanotis, at least one species of chaga, at least one species of Cordyceps, at least one species of Lycium, at least one species of reishi, at least one type of Pantocrine, at least one type of Guggulsterone, at least one species of Astragalus, at least one species of suma, at least one species of Dang Shen, at least one species of Shatavari, at least one species of Guduchi, and at least one species of Asparagus racemosus. This formulation is carefully designed using powerful herbal adaptogenic extracts that help balance hormones and the nervous system (neuroendocrine system) and HPAT (Hypothalamus Pituitary Adrenal Thyroid) Axis by reducing all types of stress while synergistically enhancing the benefits of the other two formulations in groups B and C of the base formula detailed below.

In one exemplary embodiment, the composition of group A includes at least one species of Rhaponticum uniflorum. Rhaponticum, an herbaceous perennial, has a woody horizontal rhizome, long, tough roots, and can grow to a height of over six feet. It is found in pristine alpine and subalpine zones and in the alpine meadows of southern Siberia, Kazakhstan, and the Altai region.

Rhaponticum is a classic adaptogen that includes a wide variety of anabolic actions (tissue building). It has a normalizing effect on the central nervous and cardiovascular systems; it improves sleep, appetite, moods and mental and physical states, and the functional ability of humans under stressful working conditions. It acts on the contractile proteins of the skeletal muscles as well as on other areas of the body, including the liver, heart, kidneys, and brain. It builds lean muscle, reduces body fat, improves work and athletic performance, improves mental acuity, and relieves depression. Rhaponticum is a tonic for the vital organ systems and blood and delays the effects of aging. It nourishes the HPAT axis, the endocrine hub, and is thus useful for both andropause and menopause. Rhaponticum uniflorum contains what many believe to be the world's best anabolic compound—Turkesterone. This Ecdysterone is capable of very powerful protein synthesis while at the same time reducing myostatin levels.

The amounts of Rhaponticum uniflorum in the composition may vary. However, in one exemplary embodiment, the composition contains at least 0.2 wt. % to 3 wt. % Rhaponticum uniflorum. In one exemplary embodiment, the Rhaponticum uniflorum within the composition is 40 milligrams.

In another exemplary embodiment, the composition of group A includes at least one type of Shilajit which can be used to help the body switch to an anabolic state while maintaining a healthy immune system and has been used in traditional Chinese medicine and Indian Ayurvedic medicine for its ability to restore energy, promote longevity, and improve one's quality of life.

In one exemplary embodiment, the compositions include Shilajit which is also known as Mumie, Mumiyo, Asphaltum, the sweat of mountain, balsam of rock, mineral pitch, moomiyo, mountain balsam, mountain blood, mountain rock juice, mountain tears, mummiyo.

Shilajit has been used as a rejuvenator and an adaptogen for thousands of years as part of the traditional systems of medicine in many countries. Many therapeutic properties have been ascribed to it, a number of which have been verified by modern scientific research. Shilajit possesses many miraculous healing properties. It maintains physical and mental efficiency and thus prolongs life span. It protects against illnesses and is immunomodulating. Shilajit has been used historically for general physical and mental strengthening, to delay the effects of aging, to normalize blood sugar and insulin, to enhance libido, to promote healing from injury, to enhance brain function, to support the immune system, as an antiarthritic, and for high blood pressure and obesity.

The amounts of Shilajit in the composition may vary. However, in one exemplary embodiment, the composition contains at least 1 wt. % to 7 wt. % Shilajit. In one exemplary embodiment, the Shilajit within the composition is 100 milligrams.

In another exemplary embodiment, the composition of group A includes at least one species of Rhodiola. For example, these species may include Rhodiola rosea, Rhodiola heterodonta, Rhodiola semenovii, or any combination thereof. Rhodiola (Golden Root, Roseroot) grows primarily in high mountain regions of the Arctic and North Asia, and studies have shown that Rhodiola helps improve endurance, boost mood, and fight fatigue. Species of Rhodiola such as, for example, Rhodiola rosea consists mainly of phenylpropanoids (Rosavins, rosin, rosarin (specific to R. rosea), phenylethanol derivatives (Salidroside, rhodioloside, tyrosol), flavonoids (catechins, proanthocyanidins, rhodiolin, rodionin, rodiosin, acetyl rosalgin, tricin), monoterpenes (rosiridol, rosaridin), triterpenes (daucosterol, beta-sitosterol), and phenolic acids (chlorogenic and hydroxycinnamic, gallic acids). It also contains organic acids (gallic, caffeic, and chlorogenic acids) and p-Tyrosol. There are many species of Rhodiola, but it appears that the Rosavins are unique to R. rosea, and it is the preferred species. Its therapeutic properties include a strong estrogen binding property. It also has properties of vasodilatation by activation of mu-opiate receptors in heart muscle, and it is hypolipidemic, diminishing cholesterol and triglyceride levels. The incorporation of this phytomedicine provides at least 20 active principles in a single therapeutic.

Rhodiola, used within the compositions, may be an extract. In this aspect, Rhodiola extract is prepared by extraction, preferably of the roots, and/or of the lower stem of the plant, by a solvent, followed optionally by a fractionating step enabling the active fraction(s) to be isolated. The solvent can be polar or nonpolar. Examples of solvents include, but are not limited to, pentane, decane, cyclohexane, hexane, petroleum ether, monochloromethane, dichloromethane, chloroform, isopropanol, propanol, ethyl acetate, ethanol, methanol, acetone, butylene glycol, propylene glycol, pentylene glycol, glycerol, water, and any mixture of at least two of these solvents, particularly water-alcoholic or water-glycolic mixtures.

The amounts of Rhodiola in the composition may vary. However, in one aspect the composition contains at least 1 wt. % to 8 wt. % Rhodiola. In one exemplary embodiment, the Rhodiola within the composition is 200 milligrams.

In another exemplary embodiment, the composition of group A includes at least one species of Schisandra (Schizandra). Schisandra has a long history of folkloric use as a valuable tonic and adaptogen in China, Japan, Korea, Tibet, and Russia. Traditionally, the seeds and berries were used for medicinal purposes.

Throughout the ages, many generations of people from the Far East have enjoyed the benefits of this form of Schisandra as an anti-fatigue agent. To this day, hunters in the wild in Siberia use the dry berries in the form of tea or just chew the seeds to provide extra energy during long journeys.

The amounts of Schizandra in the composition may vary. However, in one exemplary embodiment, the composition contains at least 1 wt. % to 5 wt. % Schizandra. In one aspect, the Schizandra within the composition is 115 milligrams.

In another exemplary embodiment, the composition of group A includes at least one species of Eleuthero (Acanthopanax senticosus, Russian Ginseng, Siberian Ginseng, Eleutherococcus senticosus, Devil's Shrub, Touch-me-not, Wild Pepper, Shigoka, Acantopanacis senticosus) which contains terpenoids (oleanolic acid), glycosides (Eleutherosides A (daucosterin), B1, C-G, I, K, L, M), phytosterols (β-sitosterol), coumarins (Eleutherosides B1 and B3, isofraxidine), polysaccharides (eleutherans), volatile oils, caffeic acid, coniferyl aldehyde, and sugars. Eleutherococcus is a genus of about 38 species of shrubs growing throughout Asia. Eleuthero has been shown to bind to estrogen, progestin, and mineralocorticoid receptors, and stimulate T-lymphocyte and natural killer cell production. It has activity antiplatelet aggregation activity similar to aspirin, as well as antioxidant activity. Russian Ginseng contains at least 40 active ingredients. Many members of this species contain steroidal glycosides or various chemicals that act in a similar manner to ginseng. The compositions may include at least one species of Eleutherococcus, such as Acanthopanax senticosus which includes Eleutherococcus root, stem, and leaf, extracts thereof, active components thereof, or mixtures thereof. Eleutherococcus (Acanthopanax) senticosus has been referred to as Siberian ginseng. As used herein, the preferred common name now is eleuthero. Eleuthero protects the body and enhances its various systems against the ill effects of any type of stress, including that resulting from workload, heat, cold, excessive exercise, hypokinesis, and radiation. Eleuthero demonstrates favorable effects on various human functions as well, including visual acuity, color differentiation, hearing, fatigability, and thinking in association with motor activity. More so than any other adaptogenic agent it displays a normalizing effect regardless of physiological abnormalities (e.g., normalization of blood pressure in patients with both elevated or lowered blood pressure and normalization of blood sugar levels in hyper or hypoglycemia). Eleuthero decreases adrenal hypertrophy, a common manifestation of a prolonged stress response, and spares the loss of vitamin C from the adrenal glands.

The Eleutherococcus root contains steroidal glycosides known as eleutherosides, the major compounds that provide the primary adaptogenic, antistress, anabolic actions of eleuthero, as well as ginseng. Triterpene saponins present in eleuthero include eleutherosides I, K, L, and M. Secondary constituents include phenolic compounds, chlorogenic acid being the most abundant. Also present are betulinic and caffeic acids, vitamin E, beta-carotene, sitosterol, and daucosterin, as well as a number of lignans, including sesamin and syringing. All these contribute to eleuthero's redox-antioxidative and anticancer effects. Rutoside is present in the stem bark and fruits. Minerals found in the root include calcium, phosphorus, potassium, magnesium, sodium, aluminum, barium, iron, strontium, boron, copper, zinc, manganese, and chromium. The roots are collected for extraction during the month of October when the active constituents are at their highest levels. Eleuthero leaf contains a multitude of eleutherosides, including A, B, C, D, E, F, and G; glucosides; fatty acids; and flavonoids, including anthocyanins (redox-antioxidative).

In one exemplary embodiment, examples of species of Eleutherococcus useful herein include, but are not limited to, the following: Eleutherococcus higoensis, Eleutherococcus xizangensis, Eleutherococcus scandens, Eleutherococcus brachypus, Eleutherococcus hypoleucus, Eleutherococcus rufinervis, Eleutherococcus cissifolius, Eleutherococcus cuspidatus, Eleutherococcus divaricatus, Eleutherococcus eleutheristylus, Eleutherococcus rehderianus, Eleutherococcus trichodon, Eleutherococcus stenophyllus, Eleutherococcus sieboldianus, Eleutherococcus pubescens, Eleutherococcus wardii, Eleutherococcus giraldii, Eleutherococcus henryi, Eleutherococcus huangshanensis, Eleutherococcus baoxinensis, Eleutherococcus wilsonii, Eleutherococcus japonicus, Eleutherococcus leucorrhizus, Eleutherococcus nanpingensis, Eleutherococcus nikaianus, Eleutherococcus nodiflorus, Eleutherococcus pilosulus, Eleutherococcus pseudosetulosus, Eleutherococcus senticosus, Eleutherococcus seoulensis, Eleutherococcus sessiliflorus, Eleutherococcus setchuensis, Eleutherococcus setulosus, Eleutherococcus simonii, Eleutherococcus spinosus, Eleutherococcus trifoliatus, Eleutherococcus verticillatus, Eleutherococcus lasiogyne, or any combination thereof.

The amounts of Eleuthero in the compositions may vary. However, in one exemplary embodiment, the composition contains at least 1 wt. % to 10 wt. % Eleuthero. In one aspect, the Eleuthero within the composition is 250 milligrams.

Without wishing to be bound by theory, Group A compositions above are advantageous because they provide all of the benefits of adaptogens, as described in the definition above, while each individual nutrient provides its own benefits that work together with the entire formula in a synergistic way.

Group B: An exemplary embodiment of group B (Bioenhancers) provides compositions of an herbal supplement in the form of a powder in capsules including extracts of at least three (and sometimes up to ten) agents selected from the group comprising at least one species of Ginger, at least one species of Licorice, at least one species of Pepper, at least one type of Quercetin, at least one species of Grapefruit, at least one type of Bergamotine, at least one type of Dihydroxybergamottin, at least one type of Capsaicin, at least one type of Fulvic Acid, and at least one type of Nanarin. This formulation is designed to increase the bioavailability (absorption and utilization) of all herbs, minerals, and nutrients in groups A and C of the base formula.

In an exemplary embodiment, the composition of group B includes at least one species of Ginger. Ginger is a well-known synergistic herb that potentiates, harmonizes, and improves the deep circulation and bioavailability of other herbs, reinforcing the therapeutic activity of the herb formulation. Because of its diverse therapeutic applications and a long history of use by several cultures as confirmed by modern science, ginger is classified as an adaptogen companion. Ginger influences prostaglandin metabolism as a potent inhibitor of thromboxane synthesis, significantly inhibiting platelet aggregation and inflammation. Ginger has a mild diaphoretic and antipyretic effect, making it useful for treating the flu and reducing fever. It is classified as a stimulating diaphoretic (as opposed to a relaxing diaphoretic) because it improves peripheral blood flow and does not lower the heart rate. It has recently been found to have a thermogenic effect and is thus possibly useful in weight-management programs. Ginger significantly reduces serum and hepatic cholesterol levels and possesses potent cardiotonic activity and antioxidant effects, and it is antineoplastic. Ginger also possesses remarkable proteolytic activity, making it useful for many digestive complaints. It promotes the secretion of saliva and gastric juices as well as bile. Ginger extract has been found to be as effective as ibuprofen in treating osteoarthritis. It is perhaps best known for its ability to relieve nausea.

The amounts of Ginger in the composition may vary. However, in one aspect the composition contains at least 0.5 wt. % to 2 wt. % Ginger. In one aspect, the Ginger within the composition is 25 milligrams.

In another exemplary embodiment, the composition of group B includes at least one species of Licorice. Licorice increases overall vitality while it moderates and harmonizes the characteristics of other plants to bring an herbal formula together energetically. Because of this action, in traditional Chinese medicine (TCM) it is considered to be a synergist, and it is therefore found in many classic formulas. Although there are certainly specific indications for using licorice extract as either a chief herb or a single agent (such as with Addison's disease, a hypo-functioning adrenal gland, and very low blood pressure, or certain acute inflammatory conditions), in general licorice is mostly used in small amounts in herbal formulations, wherein the amount used in the formula is usually between 2 and 5 percent of the overall. Licorice adds a soothing/cooling effect that can buffer some of the astringent, drying, and heating herbs in certain formulas. Licorice root is a classic demulcent, soothing to irritated membranes (in the lungs and GI tract), and a mild expectorant, loosening and helping with congestion in the upper respiratory tract. A number of pharmaceutical effects of licorice are known: it is anti-inflammatory, antiviral, antibacterial, antiulcer, and, among other beneficial actions.

The amounts of Licorice in the composition may vary. However, in one exemplary embodiment, the composition contains at least 0.5 wt. % to 5 wt. % Licorice. In one exemplary embodiment, the Licorice within the composition is 50 milligrams.

In another exemplary embodiment, the composition of group B includes at least one species of Pepper. Black Pepper is a source of piperine, a molecule that can inhibit enzymes that would attack other molecules. Due to this, it is ingested alongside some supplements to increase their absorption rates.

A process in the liver called glucuronidation, which attaches a molecule (glucuronide) to drugs to signal for their urinary excretion, is inhibited with piperine. This process prevents excessive levels of drugs and supplements in the body, but sometimes inhibits all uptake and renders some supplements useless. In the scenario of piperine ingestion, excretion of supplements is hindered and certain drugs and supplements can bypass this regulatory stage (as not all are subject to it).

The amounts of Pepper in the composition may vary. However, in one exemplary embodiment, the composition contains at least 0.2 wt. % to 1 wt. % Pepper. In one aspect, the Pepper within the composition is 12 milligrams.

Without wishing to be bound by theory, group B compositions above are advantageous because they provide all of the benefits of bioenhancers, as described in the definition above, while each individual nutrient provides its own benefits that work together with the entire formula in a synergistic way.

Group C: An exemplary embodiment of group C (Circulation Enhancers) provides compositions of an herbal supplement in the form of a powder in capsules including extracts of at least three (and sometimes up to twenty-four) agents selected from the group comprising at least one type of Vitamin D, at least one type of Niacin, at least one type of Magnesium, at least one type of Calcium, at least one type of Silica, at least one type of Vitamin C, at least one species of Spinach, at least one species of Hawthorn, at least one type of Cacao Polyphenol, at least one species of Pine Bark, at least one type of Capsaicin, at least one species of Artichoke, at least one species of Ginkgo, at least one species of Green Tea, at least one type of Vitamin B12, at least one type of Vitamin K2 (as menaquinone), at least one type of Arginine, at least one type of Citrulline, at least one species of Beetroot, at least one type of Nitrate, at least one type of Nitrite, at least one species of Rhubarb, at least one species of Arugula, at least one species of Butter Leaf Lettuce, and at least one species of Pepper. This formulation increases energy, stamina, oxygenation, nitric oxide levels, and circulation while boosting the benefits of the other two groups (A and B) above in the base formula.

In one exemplary embodiment, the composition of group C includes at least one type of Nitrite. Nitrite has been shown to regulate blood pressure, hypoxic vasodilation, mitochondrial efficiency, brain function, and exercise performance via the nitrate-nitrite-nitric oxide pathway. Nitric oxide (NO) is produced by nearly every type of cell in the human body and one of the most important molecules for circulation and blood vessel health. It acts as a vasodilator causing the vessels to widen. Nitric oxide production is essential for overall health because it allows blood, nutrients, and oxygen to travel to every part of the body effectively and efficiently. In this way, nitric oxide and all of its benefits are increased by nitrite.

The amounts of Nitrite in the composition may vary. However, in an exemplary embodiment, the composition contains at least 0.2 wt. % to 5 wt. % Nitrite. In an exemplary embodiment, the Nitrite within the composition is 20 milligrams.

In an exemplary embodiment, the composition of group C includes at least one type of Niacin. Niacin is an essential B-vitamin. Supplementation results in improved cholesterol and triglyceride levels. Other benefits of niacin supplementation are theorized to extend to growth, cognition, and longevity. This is because niacin supplementation increases cellular nicotinamide adenine dinucleotide (NADH/NAD+) levels. Preliminary evidence suggests increased NAD+ levels may result in the above benefits.

The amounts of Niacin in the composition may vary. However, in one aspect the composition contains at least 0.5 wt. % to 6 wt. % Niacin. In one aspect, the Niacin within the composition is 35 milligrams.

In another exemplary embodiment, the composition of group C includes at least one type of Vitamin D. Vitamin D is not technically a vitamin at, but a secosteroid molecule that has a chemical structure very similar to that of cholesterol. There are two forms of vitamin D: vitamin D3 (cholecalciferol) and vitamin D2 (ergocalciferol). Vitamin D3 is the naturally occurring and hormonally active form of vitamin D in humans, with three hydroxyl groups (abbreviated 1,25-[OH] 2 D3 or simply 1,25[OH] 2 D). Vitamin D3 is made in the skin following exposure to sunlight (UVB); it can also be found in cold-water fish (e.g., the skin of salmon) and cod liver oil. Vitamin D2 is found in some vegetables, including mushrooms, and is used to fortify foods such as commercially produced milk. D3 is the preferred form for the supplement because it is superior with regard to its ability to raise and sustain the blood and tissue level of 25(OH) vitamin D, the storage form of the vitamin. Vitamin D, which comes primarily from exposure to sunlight, promotes the absorption of calcium and phosphorus in bone mineralization, growth, and repair. Vitamin D in the food supply is usually inadequate to prevent deficiencies. It is now recognized that adequate vitamin D nutrition and sensible sun exposure during childhood not only maximizes the bone health of children but also may decrease a person's risk for many chronic diseases later in life, including type II diabetes, multiple sclerosis, rheumatoid arthritis, and common cancers. Vitamin D is a substrate (nonsteroidal hormone) for a potent, pleiotropic (producing multiple effects) repair and maintenance secosteroid hormone that serves multiple gene-regulatory functions in the body. So far, about three thousand genes have been found to be regulated by vitamin D. Optimizing vitamin D levels can also help prevent as many as sixteen different types of cancer, including pancreatic, lung, breast, ovarian, prostate, and colon cancers, as well as heart disease, diabetes, neurological disease, autoimmune disease, osteoporosis, depression, and others. It has been found that higher concentrations of 25-hydroxyvitamin D are inversely associated with all-cause and cardiovascular disease mortality among adults with hypertension in the United States. Among people that already have cancer, taking vitamin D can increase overall survival, while maintaining healthy normal levels of vitamin D can lower the risk of dying from any cause.

In healthy people, daily supplementation of vitamin D3 increased the number of circulating angiogenic myeloid cells, which are thought to promote vascular regeneration. A lack of vitamin D, even in generally healthy people, is linked with stiffer arteries and an inability of blood vessels to relax, researchers have found. In studies, participants who increased their vitamin D levels were able to improve vascular health and lower their blood pressure.

The amounts of Vitamin D in the composition may vary. However, in one aspect the composition contains at least 0.00015 wt. % to 0.00075 wt. % Vitamin D. In one aspect, the Vitamin D within the composition is 50 micrograms.

In another exemplary embodiment, the composition of group C includes at least one type of Magnesium. Magnesium is the most important major mineral in human nutrition. It is a cofactor in over 350 enzymatic reactions in the body, yet most people are depleted of this essential mineral. Magnesium participates in neuromuscular activity, including excitation-contraction coupling and cellular energy production through carbohydrate and protein metabolism. Magnesium is needed for bone strength, muscle strength, and heartbeat, for the proper functioning of the nervous system, for the correct assimilation of calcium and potassium, and for the correct and efficient functioning of many enzymatic reactions. Magnesium also competes for calcium channels and thus affects the entrance and utilization of calcium in the cells. A deficiency of magnesium can have negative consequences in hundreds of different ways because magnesium affects the mitochondria, enzymes, and membrane receptors, as well as protein, lipid, and carbohydrate synthesis. Intense aerobic and anaerobic activities cause the demand for magnesium to increase. Loss occurs via sweat and urine. Low levels of magnesium result in muscle weakness, nerve spasms, cramps, muscle spasms, and damage to the mitochondria.

Magnesium plays a major role in the health of the endothelial cells, which line vessels and allow for increased nitric oxide production and blood flow. Abnormally functioning endothelial cells are the earliest and most important change that occurs with atherosclerosis (hardening of the arteries)—hence raising stroke and heart attack risk. Magnesium reduces inflammation in blood vessels, deters platelets from adhering to the walls of blood vessels, and reduces free-radical damage to body tissues.

The amounts of Magnesium in the composition may vary. However, in one exemplary embodiment, the composition contains at least 1 wt. % to 10 wt. % Magnesium. In one aspect, the Magnesium within the composition is 80 milligrams.

Without wishing to be bound by theory, group C compositions above are advantageous because they provide all of the benefits of circulation enhancers, as described in the definition above, while each individual nutrient provides its own benefits that work together with the entire formula in a synergistic way.

### Example 1: Supportive Nutrients—Obesity Caused by a Neuroendocrine Disorder, HPAT Imbalance, and Insulin Resistance

In one exemplary embodiment the base formula containing group A (Adaptogens), group B (Bioenhancers), and group C (Circulation Enhancers) remains the same as shown above and different supportive nutrients are added to improve body composition and lipid metabolism. The exemplary embodiment provides compositions of an herbal supplement in the form of a powder in capsules including extracts of at least ten (and sometimes up to forty-three) agents selected from the group comprising at least one species of Coleus forskohlii, at least one species of Cissus quadrangularis, at least one type of Iodine, at least one type of Selenium, at least one type of Vitamin B12, at least one type of Vanadium, and at least one type of Manganese, at least one type of Chromium, at least one type of Boron, at least one species of Garcinia cambogia, at least one species of Eurycoma longifolia, at least one type of DHEA, at least one species of Tribulus terrestris, at least one type of L-dopa, at least one type of Vitamin B6, at least one species of Ashitaba, at least one type of Zinc, at least one type of Laxogenin, at least one species of Fenugreek, at least one type of Glucomannan, at least one type of Pregnenolone, at least one type of Corosolic Acid, at least one species of Irvingia gabonensis, at least one species of Shatavari, at least one species of Anacyclus pyrethrum, at least one species of White Button Mushroom, at least one species of Saw palmetto, at least one species of Green Tea, at least one species of Green Coffee Bean, at least one species of Citrus aurantium, at least one species of Gynostemma, at least one species of Dong Quai, at least one species of Lotus Leaf, at least one species of Oats, at least one type of D-aspartic acid, at least one species of Black Cohosh, at least one species of Chasteberry, at least one type of Secoisolariciresinol diglucoside, at least one type of Diindolylmethane, at least one type of 5-Hydroxytryptophan, and at least one type of Galantamine. This formulation is carefully designed using a combination of 10 or more of the herbs, minerals, and phytochemicals listed above that support healthy body composition and metabolism and synergistically improving the benefits from the base formula (groups A, B, C).

In one exemplary embodiment, the composition of additional supportive nutrients for obesity caused by neuroendocrine and HPAT disorders includes at least one species of Coleus forskohlii. Forskolin is an extract of coleus forskohlii, a member of the mint family. The compound forskolin is known to enhance adenylate cyclase, an enzyme that splits a high energy molecule of adenosine triphosphate (ATP) to yield cyclic adenosine monophosphate (cAMP). AMP causes a chain of biochemical events that pace body metabolism and food-induced thermogenesis and provide mechanisms for controlling body composition and lean body mass. The biochemical mechanism of maintaining or increasing lean body mass is related to the availability of cyclic AMP (cAMP).

By facilitating hormonal action, cAMP may regulate the body's thermogenic response to food, increase the body's basal metabolic rate, and increase utilization of body fat (since thermogenesis is preferentially fueled by fatty acids derived from body fat and/or food). These events also correspond to the buildup of lean body mass.

Forskolin bypasses the adrenergic receptors which can often decrease with age and other physiological factors and has also been shown to counteract the decreased response of fat cells to epinephrine associated with aging. Normally, cAMP is formed when a stimulatory hormone (e.g. epinephrine) binds to a receptor site on the cell membrane and triggers the activation of adenylate cyclase. Since the mechanism of action does not involve non-selective binding to the adrenergic receptors, Forskolin does not produce unwanted side effects associated with their activations.

Typically, an increase in cAMP leads to subsequent activation of protein kinase. Protein kinase has been shown to activate the enzyme lipase, which disposes of triglycerides, known as the building blocks of fatty tissue.

A clinical study showed that 25 mg of forskolin twice a day can improve overall body weight by increasing lean body mass and by decreasing weight from body fat. Therefore, forskolin can potentially benefit not only overweight people but also those individuals who are active in training or bodybuilding and are looking to increase their lean body mass to body fat ratio. This is the key benefit of forskolin, an ability to improve body composition.

In a clinical study reported in the Journal of Biological Chemistry, forskolin was also found to fully mimic the thyroid-stimulating hormone (TSH). TSH is a glycoprotein hormone secreted by the anterior lobe of the pituitary gland that stimulates and regulates the activity of the thyroid gland, which is a two-lobed endocrine gland located in front of and on either side of the trachea that produces various hormones. The thyroid gland plays a major role in determining the metabolic rate. An underactive thyroid can result in weight gain (or great difficulty losing weight), depression, fatigue, bloating and often a malaise and lack of energy, as well as cold extremities

Forskolin increases the heart rate with positive inotropic action and lowers blood pressure. Forskolin also produces cAMP-independent effects associated with the modulation of membrane transport proteins. These include adenylyl cyclase stimulation, glucose transport inhibitor, increased nicotinic receptor desensitization, and the inhibition of ligand and voltage-gated ion channels. It has been shown to inhibit platelet aggregation, further validating its beneficial cardiovascular effects. Forskolin also benefits the management of allergic reactions, the maintenance of respiratory, cardiac and circulatory health, glaucoma, ocular hypertension, psoriasis, and is a safe and effective therapeutic approach in the management of vasculogenic impotence that is resistant to standard pharmacotherapy.

In a study of overweight women who were given Forskolin for eight weeks, mean values for body weight and fat content significantly decreased, whereas lean body mass was significantly increased as compared to the baseline. A trend was observed of lower systolic/diastolic pressure during the course of treatment.

The amounts of Forskolin in the composition may vary. However, in one exemplary embodiment, the composition contains at least 0.01 wt. % to 0.04 wt. % Forskolin. In one exemplary embodiment, the Forskolin within the composition is 20 milligrams.

In another exemplary embodiment, the composition of additional supportive nutrients includes at least one species of Cissus quadrangularis. Cissus quadrangularis is a perennial plant of the grape family. It is commonly known as veldt grape, devil's backbone, adamant creeper, asthisamharaka, hadjod, and pirandai. The species is native to tropical Asia, Arabia and much of Africa.

Cissus quadrangularis is an evergreen climber growing to 16 ft by 1.6 ft at a fast rate. It is hardy to zone 10. Suitable for: light (sandy), medium (loamy) and heavy (clay) soils, prefers well-drained soil and can grow in nutritionally poor soil. Suitable pH: acid, neutral and basic (alkaline) soils and can grow in very acidic and very alkaline soils. It cannot grow in the shade. It prefers dry or moist soil and can tolerate drought.

One preliminary clinical study found a benefit in weight reduction and an improvement in the symptoms associated with metabolic syndrome in obese patients who had been given C. quadrangularis supplements. Another study found a potential synergistic effect between C. quadrangularis and Irvingia gabonensis. A weight loss supplement containing Cissus quadrangularis and other ingredients including green tea, soy, selenium, chromium, and B vitamins was evaluated in an 8-week trial. The supplement helped reduce body weight by 4-8% (placebo 2.4%) a clinically significant weight loss.

The amounts of Cissus quadrangularis in the composition may vary. However, in one aspect the composition contains at least 3 wt. % to 7 wt. % Cissus quadrangularis. In one aspect, the Cissus quadrangularis within the composition is 200 milligrams.

In another aspect, the composition of additional supportive nutrients includes at least one form of Iodine. Iodine is a naturally occurring mineral found in igneous rocks at 0.5 ppm. Iodine's main role is to help manufacture thyroid hormones. Some 11 million Americans have either a hypothyroid (low, underactive) or a hyperthyroid (overactive) condition. Thyroid hormones control and regulate energy levels, digestion, heart rate, body temperature, sweat gland activity, nervous and reproductive system, general metabolism, and body weight. Iodine intake has dropped by around 50 percent in the last 30 years and related compounds added to food and water (bromide, halides, chlorine, and fluorine) may actually block the activity of iodine.

The amounts of Iodine in the composition may vary. However, in one exemplary embodiment, the composition contains at least 0.01 wt. % to 0.04 wt. % Iodine. In one aspect, the Iodine within the composition is 150 micrograms.

In another aspect, the composition of additional supportive nutrients includes at least one type of Selenium. Selenium is a naturally occurring mineral found in igneous rocks at 0.05 ppm. Selenium is an essential trace element that provides antioxidant protection in concert with vitamin E. Several selenoproteins have been identified, but only one, glutathione peroxidase, a selenoenzyme, has a known function, that of neutralizing toxic lipid hydroperoxides. Selenium, however, has several metabolic effects not associated with glutathione peroxidase. Selenium prevents cellular and subcellular lipids and fats from being peroxidized. For proper thyroid function, a selenium-dependent enzyme is needed to convert T4 (the inactive form of thyroid hormone) to T3 (the active form). In young adults, selenium deficiency appears as anemia, chronic fatigue, hypothyroidism, liver cirrhosis, muscular weakness, myalgia, muscle tenderness, fibromyalgia, lupus, pancreatitis, and infertility. Selenium also has immunopotentiating properties. It is required for optimal macrophage and natural killer cell functions.

The amounts of Selenium in the composition may vary. However, in one exemplary embodiment, the composition contains at least 0.1 wt. % to 0.4 wt. % Selenium. In one exemplary embodiment, the Selenium within the composition is 100 micrograms.

In another exemplary embodiment, the composition of additional supportive nutrients includes at least one type of Vitamin B12. Vitamin B12 is mostly found in animal-based foods such as meat, fish, eggs, and cheese and is an important cofactor in the enzymatic processes that produce nitric oxide which is responsible for improving vascular function and energy levels. In fact, one of the most common early signs of vitamin B12 deficiency is fatigue or lack of energy. Vitamin B12 plays a vital role in helping your body produce red blood cells. If you're significantly deficient in vitamin B12, your homocysteine levels become elevated. High blood levels of the common amino acid homocysteine have been linked to an increased risk of heart disease and mental dysfunction. Vitamin B12 also plays a vital role in synthesizing and metabolizing serotonin, a chemical responsible for regulating mood and depression.

The amounts of Vitamin B12 in the composition may vary. However, in one exemplary embodiment, the composition contains at least 0.01 wt. % to 0.04 wt. % Vitamin B12. In one exemplary embodiment, the Vitamin B12 within the composition is 500 micrograms.

In another exemplary embodiment, the composition of additional supportive nutrients includes at least one type of Vanadium. Vanadium compounds increase glucose transport activity and improve glucose metabolism. It is also associated with greater insulin sensitivity. Vanadium compounds mimic many of the metabolic actions of insulin both in vitro and in vivo and improve glycemic control in humans with diabetes mellitus. It also modulates insulin metabolic effects by enhancing insulin sensitivity and prolonging insulin action. The ability of vanadium compounds to ‘bypass’ defects in insulin action in diseases characterized by insulin resistance and they're apparent preferential metabolic versus mitogenic signaling profile make them attractive as potential pharmacological agents. Vanadium has demonstrated antihypertensive and anorectic properties as well.

The amounts of Vanadium in the composition may vary. However, in one exemplary embodiment, the composition contains at least 0.4 wt. % to 0.9 wt. % Vanadium. In one exemplary embodiment, the Vanadium within the composition is 250 micrograms.

In another exemplary embodiment, the composition of additional supportive nutrients includes at least one type of Manganese. Manganese is found in igneous rocks at 950 ppm; shale at 850 ppm; sandstone at 50 ppm; limestone at 1,100 ppm; marine plants at 1 to 60 ppm (lowest in fish); land animals at 0.2 ppm (highest in concentrations in mammalian liver and kidney); the total body content of Mn in humans is only 10 to 20 milligrams.

Manganese is essential to all known living organisms; it activates numerous enzyme systems including those involved with glucose metabolism, energy production, and superoxide dismutase; it is a major constituent of several metalloenzymes, hormones, and proteins of humans.

Manganese plays a role in many enzymes, including those involved in blood sugar, energy production, and thyroid hormones. But it's particularly important for bone health as it helps form collagen (the protein substructure for minerals in the bone) and also helps remineralize bones. Few Americans have an adequate intake of this nutrient.

The amounts of Manganese in the composition may vary. However, in one exemplary embodiment, the composition contains at least 0.2 wt. % to 1 wt. % Manganese. In one exemplary embodiment, the Manganese within the composition is 2 milligrams.

In another exemplary embodiment, the composition of additional supportive nutrients includes at least one type of Chromium. Chromium is a natural mineral found in igneous rock at 100 ppm. Chromium is an essential trace element that is responsible for producing a substance called glucose tolerance factor (GTF). GTF is important in the utilization of insulin, a hormone that stabilizes blood sugar levels and is critical to the metabolism and storage of carbohydrates, fats, cholesterol, and proteins in the body. The mineral Chromium increases the body's ability to use insulin by increasing the number of insulin receptors, boosting an enzyme that helps those receptors work, and then blocking an enzyme that turns the receptors off. Poor diet, acute exercise, recent infections, pregnancy/lactation, and physical trauma can lead to chromium depletion, thus impairing the body's ability to utilize glucose efficiently.

Normal dietary intake of chromium for humans is suboptimal. The estimated safe and adequate daily dietary intake for chromium is 50 to 800 mcg. Most diets contain less than 60% of the minimum suggested intake of 50 micrograms. Insufficient dietary intake of chromium leads to signs and symptoms that are similar to those observed for diabetes and cardiovascular disease. Supplemental chromium given to people with impaired glucose tolerance or diabetes leads to improved blood glucose, insulin, and lipid variables.

Chromium levels deplete with age and illness, as well as with exercise. Such depletion, when it occurs with excessive consumption of sugar and other carbohydrates, may result in glucose intolerance, glycosuria, hyperinsulinemia, and hyperlipidemia. The key benefit of chromium is in regulating and facilitating the efficient function of insulin. Chromium potentiates the action of insulin in vitro and in vivo. As a cofactor in insulin utilization, chromium maintains proper blood sugar levels.

In a randomized, double-blind placebo-controlled clinical trial conducted at Wake Forest University, insulin resistance was reduced by a statistically significant 40% and this improvement was maintained at the end of eight months. The placebo group gained 6.5% abdominal fat while the chromium group gained just 1%.

Chromium is an essential nutrient involved in the regulation of carbohydrate lipid metabolism and can break the cycle of overeating sweets. Eating sugar and high-fructose food and beverages causes chromium deficiency which can simultaneously cause drastic increases in insulin and glucose levels. This will trigger a person's craving for fattening sweets, which causes the cycle to repeat. When insulin is functioning efficiently, blood sugar and fatty acids metabolize properly, producing heat (thermogenesis) instead of weight gain.

In a second clinical study, chromium supplementation increased lean body mass in obese patients. Dermatologists report that chromium appears to have value as an acne treatment.

The amounts of Chromium in the composition may vary. However, in one aspect the composition contains at least 0.5 wt. % to 1 wt. % Chromium. In one exemplary embodiment, the Chromium within the composition is 400 micrograms.

In another exemplary embodiment, the composition of additional supportive nutrients includes at least one species of Eurycoma longifolia. Eurycoma (Tongkat Ali) comes from a tree found in the jungles of Malaysia, Indochina, Borneo, and Sumatra and has been used for thousands of years in indigenous traditional medicines throughout Malaysia. It has been shown to reduce blood pressure, fevers, and fatigue, and in some studies, it has shown an ability to enhance free testosterone levels, athletic performance, libido, energy, muscle mass, and vitality.

The amounts of Eurycoma in the composition may vary. However, in one exemplary embodiment, the composition contains at least 2.5 wt. % to 14 wt. % Eurycoma. In one exemplary embodiment, Eurycoma longifolia within the composition is 300 milligrams.

### Example 2: Supportive Nutrients—Supporting Cardiovascular Health

In one exemplary embodiment the base formula containing group A (Adaptogens), group B (Bioenhancers), and group C (Circulation Enhancers) remains the same as shown above and different supportive nutrients are added for cardiovascular health. The invention provides compositions of an herbal supplement in the form of a powder in capsules including extracts of at least ten (and sometimes up to twenty-five) agents selected from the group comprising at least one type of Molybdenum, at least one type of Astaxanthin, at least one type of Huperzine A, at least one species of Garlic, at least one species of Artichoke, at least one type of Vitamin K2, at least one type of Vitamin C, at least one type of Vitamin E, at least one type of Coenzyme Q10, at least one type of Nicotinamide Riboside (NR), at least one type of Nicotinamide Mononucleotide (NMN), at least one type of Sodium Hydrosulfide, at least one species of Arjuna, at least one type of Lipoic Acid, at least one type of Folate, at least one type of Quercetin, at least one species of Guggul, at least one species of Green Tea, at least one type of Policosanol, at least one species of Cinnamon Bark, at least one species of Motherwort, at least one type of Chrysin, at least one species of Goat's Rue, at least one species of Green Coffee Bean, at least one species of Grape Seed, at least one species of Hawthorn, at least one type of Bromelain, at least one species of Linden Flower, at least one type of Curcumin, at least one type of Turmeric, at least one type of NADH/NAD+(Nicotinamide Adenine Dinucleotide), at least one type of Bitter Melon, at least one type of Resveratrol, and at least one type of Nattokinase or Lumbrokinase. This formulation is carefully designed using a combination of 10 or more of the herbs, minerals, and phytochemicals listed above that support a healthy cardiovascular system and synergistically improving the benefits from the base formula (groups A, B, C).

In one exemplary embodiment, the composition of additional supportive nutrients for cardiovascular health includes at least one type of Lipoic Acid. Lipoic Acid facilitates the recycling of the antioxidant vitamins E and C in the body, promotes the production of glutathione, and supports glucose and insulin utilization. It also aids cysteine in glutathione regulation.

The amounts of Lipoic Acid in the composition may vary. However, in one aspect the composition contains at least 0.8 wt. % to 2.5 wt. % Lipoic Acid. In one aspect, the Lipoic Acid within the composition is 50 milligrams.

In another exemplary embodiment, the composition of additional supportive nutrients includes at least one species of Hawthorn (Crataegus oxyacantha). Hawthorn contains mainly flavonoids (such as flavonoglycosyls, hyperoside, rutin, flavonol, kaempferol, and quercetin) and oligomeric procyanidins (1-epicatechol), which relax arterial expansion to decrease peripheral vascular resistance. It also contains amines (phenylethylamine, tyramine, O-methoxy phenethylamine), flavones (apigenin, luteolin) derivatives, vitexin glycosides, tannins, saponins, and cyanogenic glycosides. The incorporation of this phytomedicine into the composition provides more than 50 active principles in a single therapeutic plant.

The amounts of Hawthorn in the composition may vary. However, in one exemplary embodiment, the composition contains at least 1 wt. % to 4 wt. % Hawthorn. In one exemplary embodiment, the Hawthorn within the composition is 100 milligrams.

In another exemplary embodiment, the composition of additional supportive nutrients includes at least one type of NADH/NAD+ (Nicotinamide Adenine Dinucleotide). NADH, a natural coenzyme derived from niacin (vitamin B3) is essential for basic metabolism, respiration, the breakdown of sugars and fats, as well as the production of ATP, the primary energy molecule in our cells. NADH supplies ATP energy to the brain, nerves, muscles, heart and all other organs.

NADH is a coenzyme found in all living cells. It is a dinucleotide, which means that it consists of two nucleotides joined through their phosphate groups. One nucleotide contains an adenine base, and the other contains nicotinamide. In metabolism, NADH helps facilitate cellular functions, DNA repair, growth, and many more things. Quite simply, without NADH, life would not be possible.

The amounts of NADH in the composition may vary. However, in one aspect the composition contains at least 1 wt. % to 4 wt. % NADH. In one aspect, NADH within the composition is 100 milligrams.

In another exemplary embodiment, the composition of additional supportive nutrients includes at least one type of (NMN) Nicotinamide Mononucleotide. Not long ago, this molecule was only known for its activity as an intermediate in nicotinamide adenine dinucleotide (NAD+) biosynthesis. More recent research in human cells has shown NMN is available as a source of cellular energy. NMN has also been studied in animal models as a potential neuroprotective and anti-aging agent.

The amounts of NMN in the composition may vary. However, in one aspect the composition contains at least 2 wt. % to 6 wt. % NMN. In one aspect, the NMN within the composition is 100 milligrams.

In another exemplary embodiment, the composition of additional supportive nutrients includes at least one type of NR (Nicotinamide Riboside). NR is an alternative form of vitamin B3 (niacin). Like other forms of vitamin B3, NR is converted by your body into nicotinamide adenine dinucleotide (NADH), a coenzyme or helper molecule. NADH acts as fuel for many key biological processes, such as converting food into energy, repairing damaged DNA, fortifying cells' defense systems, setting your body's internal clock or circadian rhythm.

The amounts of NR in the composition may vary. However, in one exemplary embodiment, the composition contains at least 2 wt. % to 6 wt. % NR. In one aspect, the NR within the composition is 100 milligrams.

In another exemplary embodiment, the composition of additional supportive nutrients includes at least one type of Vitamin K2. Vitamin K is an essential vitamin that is known for helping maintain calcium balance in the body. Supporting everything from arterial health to healthy bone density, vitamin K is an important part of maintaining cardiovascular health.

The amounts of Vitamin K2 in the composition may vary. However, in one aspect the composition contains at least 0.0003 wt. % to 0.0013 wt. % Vitamin K2. In one aspect, the Vitamin K2 within the composition is 50 micrograms.

In another exemplary embodiment, the composition of additional supportive nutrients includes at least one type of Huperzine A. Huperzine A acts as a cholinesterase inhibitor that works by improving the levels of neurotransmitters in the brain.

Acetylcholine is a neurotransmitter and neuromodulator. It plays an important role in cognitive function, most notably, in the neural mechanisms of memory. In addition to this memory function, acetylcholine is involved in supporting alertness, attention, and learning.

One of Huperzine A's most recognized biological actions is its involvement in acetylcholine signaling. Huperzine A has improved acetylcholine levels in animal and human studies and influences cholinesterase enzyme activity. The net effect is an increase in acetylcholine. This involvement with acetylcholine signaling might explain its reported benefits on memory and cognition

The amounts of Huperzine A in the composition may vary. However, in one exemplary embodiment, the composition contains at least 0.3 wt. % to 1 wt. % Huperzine A. In one exemplary embodiment, Huperzine A within the composition is 50 mcg.

In another exemplary embodiment, the composition of additional supportive nutrients includes at least one type of Astaxanthin. Astaxanthin has been shown to relieve general eye fatigue and promote a healthy inflammatory response. Astaxanthin helps optimize immune, memory, cardiovascular, metabolic, and mitochondrial health. It can even help maintain healthy cognitive function and encourage muscle strength and endurance.

The amounts of Astaxanthin in the composition may vary. However, in one exemplary embodiment, the composition contains at least 0.5 wt. % to 2 wt. % Astaxanthin. In one exemplary embodiment, the Astaxanthin within the composition is 20 milligrams.

In another exemplary embodiment, the composition of additional supportive nutrients includes at least one type of Linden Flower. Linden flowers have been used in traditional herbalism for colds, cough, fever, infections, inflammation, high blood pressure, and headache (particularly migraine), and as a diuretic, antispasmodic (reduces smooth muscle spasm along the digestive tract), and sedative. In traditional Austrian medicine, the flowers have been used internally as a tea for the treatment of disorders of the respiratory tract, fever, and flu. New evidence suggests that the flowers may be hepatoprotective.

The amounts of Linden Flower in the composition may vary. However, in one exemplary embodiment, the composition contains at least 1 wt. % to 4 wt. % Linden Flower. In one exemplary embodiment, Linden Flower within the composition is 100 milligrams.

In another exemplary embodiment, the composition of additional supportive nutrients includes at least one type of Resveratrol. Resveratrol is a compound most commonly found in red wine and grapes, as well as in some plants like Japanese knotweed. Researchers have observed better heart health in populations that consume a diet that emphasizes healthy fats and moderate amounts of red wine. Resveratrol supports the cardiovascular system, encouraging endothelial health and promote the expression of genes associated with healthy aging. These so-called longevity genes encourage the healthy structure and function of cells and organs throughout the body. Additionally, Resveratrol encourages healthy insulin sensitivity, supports healthy mitochondrial function, and helps inhibit inflammatory factors to promote overall health and longevity.

The amounts of Resveratrol in the composition may vary. However, in one exemplary embodiment, the composition contains at least 1 wt. % to 4 wt. % Resveratrol. In one exemplary embodiment, the Resveratrol within the composition is 50 milligrams.

### Example 3: Supportive Nutrients—Supporting Brain Health

In one exemplary embodiment the base formula containing group A (Adaptogens), group B (Bioenhancers), and group C (Circulation Enhancers) remains the same as shown above and different supportive nutrients are added to improve memory and brain health. The invention provides compositions of an herbal supplement in the form of a powder in capsules including extracts of at least at least ten (and sometimes up to sixty nine) agents selected from the group comprising at least one species of Ginkgo biloba, at least one species of Gotu Kola, at least one species of Saffron, at least one species of Kanna, at least one type of Mildronate, at least one species of Rosemary, at least one species of Lemon Balm, at least one type of Methylliberine, at least one species of Lion's Mane, at least one species of Bacopa monnieri, at least one species Pine Bark, at least one type of Phenylpiracetam, at least one species of Passionflower, at least one type of Folate, at least one species of Bilberry, at least one type of Resveratrol, at least one type Silk Protein Hydrolysate, at least one type of 7,8 Dihydroxyflavone, at least one type of Nicotinamide Riboside (NR), at least one type of Nicotinamide Mononucleotide (NMN), at least one type of Nicotinamide Adenine Dinucleotide (NADH), at least one species of Celastrus paniculatus, at least one type of Lithium, at least one type of PQQ Disodium Salt, at least one type of Uridine, at least one type of Modafinil, at least one type of Centrophenoxine, at least one type of Tianeptine Sulfate, at least one species of Sage, at least one type of Vinpocetine, at least one type of Galantamine, at least one type of Phosphatidylserine (PS), at least one type of Sulbutiamine, at least one type of Nicotine, at least one type of Gamma-Aminobutyric Acid (GABA), at least one type of Semax, at least one type of Fasoracetam, at least one type of Citicoline, at least one type of Nattokinase, at least one type of Adrafinil, at least one type of Pterostilbene, at least one type of Diindolylmethane (DIM), at least one type of Lipoic Acid, at least one type of Coenzyme Q10, at least one type of Huperzine A, at least one type of Hesperidin, at least one type of Curcumin, at least one type of Theacrine, at least one type of Choline, at least one type of Bromantane, at least one type of Inositol, at least one type of Methionine, at least one type of L-Theanine, at least one type of Brain-Derived Neurotrophic Factor (BDNF), at least one type of L-Tyrosine, at least one type of Carnitine, at least one type of Phycocyanin, at least one type of DHA (Docosahexaenoic acid), at least one type of Boron, at least one type of Pyritinol, at least one type of Coluracetam, at least one type of Zinc, at least one type of Vitamin B6, at least one type of 5-HTP, at least one type of Iodine, at least one type of Noopept, at least one type of Magnesium, at least one type of Waklert, and at least one type of L-Dopa. This formulation is carefully designed using a combination of 10 or more of the herbs, minerals, and phytochemicals listed above that support healthy brain function and synergistically improving the benefits from the base formula (groups A, B, C).

In one aspect, the composition of additional supportive nutrients for brain health includes at least one type of L-dopa. Mucuna pruriens is an annual climbing legume that grows three to eighteen meters high. It is indigenous to tropical regions, especially Africa, India, and the West Indies. The plant produces clusters of pods that contain seeds known as Mucuna beans. These seedpods are covered with reddish-orange hairs that are readily dislodged and can cause intense irritation to the skin. This is where the name pruriens, Latin for “itching sensation,” comes from. Mucuna pruriens is a rich source of the amino acid L-dopa, which is the precursor to the neurotransmitters dopamine, norepinephrine (noradrenaline), and epinephrine (adrenaline), collectively known as catecholamines. Dopamine regulates motor control, sex drive, immune function, growth hormone levels, and growth hormone release, and motivational behavior. Low dopamine levels in the brain cause a decline in hypothalamic-releasing factors, which hinders the ability of the hypothalamic-pituitary axis to stimulate testosterone release in both men and women. Mucuna pruriens seed powder not only reactivates the enzymatic activity of metabolic pathways and energy metabolism, but it also rejuvenates the harmonic balance of male reproductive hormones in infertile men.

Mucuna pruriens is a leguminous plant member of the Fabaceae family. Mucuna pruriens grows pods about twelve centimeters long that contain about seven seeds of varied coloration from beige to brown and black or having stripes. Also known as velvet bean, lion bean, Nescafe, and cowage, Mucuna pruriens originated in India and is now grown in Sri Lanka, Malaysia, southeastern Asia, Brazil, and tropical regions of Central and South America. Studies indicate that Mucuna pruriens has aphrodisiac properties, improves fertility and muscle mass, includes various sterols, and has high levels of L-dopa, an amino acid manufactured by the human body and a precursor to dopamine.

L-dopa is an amino acid and a hormone that is made naturally by a number of plants and animals. In humans, it is created via biosynthesis from the amino acid L-Tyrosine. It is a precursor to several important and powerful neurotransmitters which include dopamine, norepinephrine (noradrenaline) and epinephrine (adrenaline). These are collectively known as catecholamines and play a role in the stimulation and arousal systems in your brain, including stimulation and arousal related to sexual performance. In its pure form, L-dopa is considered to be a psychoactive chemical, and can also be used for the treatment of Parkinson's disease and a number of other conditions related to lowered levels of the above neurotransmitters (especially dopamine). As a nootropic, L-dopa is used to combat depression and anxiety while enhancing mood and improving the ability to concentrate and focus.

L-dopa causes a stimulatory effect on the human central nervous system resulting in the hypothalamus area of the brain, along with the pituitary gland, releasing increased levels of growth hormone (HGH). As a precursor, L-dopa has also been shown to increase the levels and activities of the adrenergic hormones norepinephrine and epinephrine. The combined presence of these increased levels of hormones and neurotransmitters are responsible for a number of beneficial effects like enhanced motor control, sex drive, immune function, fat loss (and gain), lean muscle growth, bone density, and energy level.

The amounts of Mucuna pruriens (L-Dopa) in the composition may vary. However, in one exemplary embodiment, the composition contains at least 4.5 wt. % to 6 wt. % L-Dopa. In one exemplary embodiment, the L-Dopa within the composition is 150 milligrams.

In another exemplary embodiment, the composition of additional supportive nutrients includes at least one type of Sage. Sage has been traditionally used for digestive problems, including loss of appetite, gas (flatulence), stomach pain (gastritis), diarrhea, bloating, heartburn, depression, memory loss, and Alzheimer's disease. Some research has shown that taking a single dose of common sage (Salvia officinalis) or Spanish sage (Salvia lavandulaefolia) by mouth seems to improve memory, alertness, and attention in healthy adults.

The amounts of Sage in the composition may vary. However, in one exemplary embodiment, the composition contains at least 1 wt. % to 4 wt. % Sage. In one exemplary embodiment, the Sage within the composition is 100 milligrams.

In another exemplary embodiment, the composition of additional supportive nutrients includes at least one type of Zinc. Zinc is a trace element that is necessary for a healthy immune system. A lack of zinc can make a person more susceptible to disease and illness. It is responsible for a number of functions in the human body, and it helps stimulate the activity of at least 100 different enzymes. Only a small intake of zinc is necessary to reap the benefits. Currently, the recommended dietary allowance (RDA) for zinc in the United States is 8 milligrams (mg) a day for women and 11 mg a day for men.

The amounts of Zinc in the composition may vary. However, in one aspect the composition contains at least 0.3 wt. % to 5 wt. % Zinc. In one aspect, the Zinc within the composition is 5 milligrams.

In another exemplary embodiment, the composition of additional supportive nutrients includes at least one type of Lion's Mane. Revered in many ancient cultures and used medicinally for thousands of years, Lion's Mane fungi has been used traditionally for everything from protecting the brain against cognitive decline to boosting memory, supporting immunity, reducing anxiety, boosting mood and enhancing a sense of focus throughout your day, among many other things.

The amounts of Lion's Mane in the composition may vary. However, in one exemplary embodiment, the composition contains at least 3 wt. % to 7 wt. % Lion's Mane. In one exemplary embodiment, the Lion's Mane within the composition is 200 milligrams.

In another exemplary embodiment, the composition of additional supportive nutrients includes at least one type of Bacopa monnieri. Bacopa monnieri has been used by Ayurvedic medical practitioners for centuries for a variety of purposes, including improving memory, reducing anxiety, treating epilepsy, alleviate anxiety and stress, among other benefits.

The amounts of Bacopa monnieri in the composition may vary. However, in one exemplary embodiment, the composition contains at least 3 wt. % to 7 wt. % Bacopa monnieri. In one exemplary embodiment, Bacopa monnieri within the composition is 200 milligrams.

In another exemplary embodiment, the composition of additional supportive nutrients includes at least one type of Huperzine A. Huperzine A acts as a cholinesterase inhibitor that works by improving the levels of neurotransmitters in the brain.

Acetylcholine is a neurotransmitter and neuromodulator. It plays an important role in cognitive function, most notably, in the neural mechanisms of memory. In addition to this memory function, acetylcholine is involved in supporting alertness, attention, and learning.

One of Huperzine A's most recognized biological actions is its involvement in acetylcholine signaling. Huperzine A has improved acetylcholine levels in animal and human studies and influences cholinesterase enzyme activity. The net effect is an increase in acetylcholine. This involvement with acetylcholine signaling might explain its reported benefits on memory and cognition

The amounts of Huperzine A in the composition may vary. However, in one aspect the composition contains at least 0.3 wt. % to 1 wt. % Huperzine A. In one aspect, Huperzine A within the composition is 50 mcg.

In another exemplary embodiment, the composition of additional supportive nutrients includes at least one type of Lithium. Lithium's neuroprotective benefits may reduce brain shrinkage, thus reducing your risk of age-related diseases. It is also known to boost cognitive function and strengthen your ability to focus. In addition to being helpful for balancing mood and boosting brain health, lithium may offer many other benefits including heart health, bone health, and the immune system health.

The amounts of Lithium in the composition may vary. However, in one exemplary embodiment, the composition contains at least 0.1 wt. % to 0.5 wt. % Lithium. In one exemplary embodiment, the Lithium within the composition is 5 milligrams.

In another exemplary embodiment, the composition of additional supportive nutrients includes at least one type of Centrophenoxine. Centrophenoxine is a cholinergic compound with DMAE (Dimethylaminoethanol) component and acts as a better-absorbed transporter of DMAE into the brain. It is effective in reversing some of the signs of aging (particularly waste product buildup in the brain) when taken for an acute period (high doses for a month) and can act as a general neural enhancer and protector when taken continuously at a lower dose.

The amounts of Centrophenoxine in the composition may vary. However, in one exemplary embodiment, the composition contains at least 1 wt. % to 5 wt. % Centrophenoxine. In one exemplary embodiment, the Centrophenoxine within the composition is 50 milligrams.

In another exemplary embodiment, the composition of additional supportive nutrients includes at least one type of Phosphatidylserine (PS). Phosphatidylserine is an amino acid derivative compound that is fat-soluble and found in high amounts in the brain, where it contributes to cognitive functioning. Found in high amounts in fish, it may improve memory in the elderly and lower cortisol.

Phosphatidylserine, or PS, is similar to a dietary fat that is highly prevalent in human neural tissue. It can be synthesized as well as consumed through the diet, but further benefits can be gained through supplementation.

The amounts of Phosphatidylserine in the composition may vary. However, in one exemplary embodiment, the composition contains at least 1 wt. % to 5 wt. % Phosphatidylserine. In one exemplary embodiment, Phosphatidylserine within the composition is 50 milligrams.

In another exemplary embodiment, the composition of additional supportive nutrients includes at least one type of Vinpocetine. Vinpocetine is a synthetic alkaloid derived from the periwinkle plant (specifically, synthesized from the molecule known as ‘vincamine’) that has a track record of usage in European countries for the treatment of cognitive decline, stroke recovery, and epilepsy. Vinpocetine is also commonly used as a nootropic compound in the hopes that it may promote memory formation.

Vinpocetine appears to have a role in neuroprotection and reducing neural inflammation, and for healthy persons, it may potentially increase blood flow to the brain and improve reaction time.

The amounts of Vinpocetine in the composition may vary. However, in one exemplary embodiment, the composition contains at least 0.1 wt. % to 0.8 wt. % Vinpocetine. In one exemplary embodiment, the Vinpocetine within the composition is 10 milligrams.

### Exemplary Formulations

An example of an herbal extract in accordance with exemplary embodiments, and of its method of manufacture is set forth below.

### EXAMPLE

In the exemplary embodiments, the dietary supplement comprises various nutraceuticals, phytochemicals, vitamins, minerals, phytoceuticals and/or synthetic equivalents. The plant ingredients from the plants described above are in the form of plant extracts, derivatives thereof, or synthetic equivalents. A plant extract may be taken from the leaves, roots, stem, flower, seeds or any other component of the plant, as appropriate, to provide a maximum therapeutic effect. Any of the plant extracts or derivatives thereof may be substituted with an active constituent or synthetic equivalent that is believed to contribute to the improvement or enhancement of the particular formula.

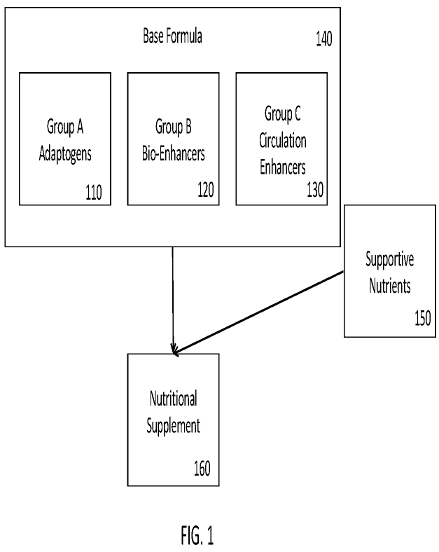

Referring to FIG. 1, as stated above, formulations of a nutritional supplement 150 may be formed by combining ingredients from Group A Adaptogens 110, Group B Bioenhancers 120, and Group C Circulation Enhancers 130 in a Base Formula 140. Base Formula 140 is combined with Supportive Nutrients 150 in a manufacturing process to result in a Nutrient Supplement 160. Nutrient Supplement 160 may be manufactured in any of a variety of forms, as discussed herein, for use by a consumer.

A preferred all-natural herbal supplement of an exemplary embodiment is manufactured in a cGMP compliant facility. Manufacturing begins by admixing the ingredients set forth below in the relative amounts set forth. Thereafter the ingredients undergo encapsulation and bottling in accordance with the technique set forth infra.

In one exemplary embodiment, 1 weight % part is 1 mg.
Sample formulation to reduce obesity caused by a neuroendocrine disorder, HPAT imbalance, and insulin resistance:

### Ashwagandha Root Ext (10% Withanolides)—150 mg

### Rhodiola Root Ext (1% Rosavins & 5% Salidroside)—300 mg

### Schizandra Ext (9% Schizandrins)—115 mg

Rhaponticum uniflorum Ext (40% Turkesterone)—10 mg
Panax ginseng Ext (40% Ginsenosides)—100 mg

### Shilajit Ext (50% Fulvic Acid)—200 mg

### Eleuthero Root Ext (0.5% E & 1.5% B Eleutherosides)—300 mg

### Ginger Root Ext (20% Gingerols)—25 mg

### Licorice Root Ext (20% Glycyrrhizin)—50 mg

### Black Pepper Ext (95% Piperine)—12 mg

### Niacin (as Nicotinic Acid)—40 mg

Nitrite (from Sodium Nitrite)—40 mg

### Vitamin C (as Calcium Ascorbate)—50 mg

### Vitamin D3 (as Cholecalciferol)—50 mcg

### Magnesium (as Magnesium Creatine Chelate)—5 mg

Mucuna pruriens Seed Ext (98% L-Dopa)—150 mg
Tribulus terrestris Fruit Ext (40% Protodioscin & 95% Saponins)—40 mg

### Tongkat Ali Root Ext (8% Eurycomanone)—25 mg

### Tongkat Ali Root Ext (200:1)—400 mg

DIM (diindolylmethane)—100 mg

### Boron (as Bororganic Glycine)—10 mg

### Zinc (as Zinc Bisglycinate Chelate)—10 mg

Coleus forskohlii Ext (20% Forskolin)—20 mg

### Loquat Leaf Ext (10% Corosolic Acid)—4 mg

Griffonia simplicifolia Ext (5-HTP)—100 mg

### Iodine (as Potassium Iodide)—150 mcg

### Selenium (as Selenium Glycinate)—100 mcg

### Vanadium (as Vanadium Nicotinate Glycinate Chelate)—250 mcg

### Vitamin B12 (as Methylcobalamin)—500 mcg

### Chromium—500 mcg

### Example Plant Extraction Process

Place the pre-mixed herbal material (1 kg) in a round bottom flask of appropriate size, add 3 liters of 50% ethanol (the other 50% water). Carry out heated extraction under reflux for an additional 2 hours. Cool the content in the flask to room temperature and pour out the ethanol solution (Solution I). To the herb residue in the flask, add 2 liters of 50% ethanol and reflux for an additional 2 hours. Cool the content in the flask to room temperature and pour out the ethanol solution (Solution II). Combine Solutions I and II. Recover ethanol from the combined solution by distillation to reduce the volume of the solution to about 2.5 liters. Adjust the ethanol content to around 20% and the volume to 3.5 liters and stand at room temperature for 24 hours. Filter through cotton and wool. Add 20% ethanol to the filtrate to make the final volume to one US Gallon. Next spray dry the solution to make a fine powder that can be used in capsules, tablets, and other delivery methods.

### Usage of an Exemplary Embodiment:

The recommended daily dosage of the foregoing formulation is between 1000 mg and 6000 mg each day depending on body weight. As a dietary supplement, take 2 capsules two to three times daily with food (total of 4-6 capsules daily).

This formula is intended to be offered as a Dietary Supplement under the Dietary Supplement Health and Education Act of 1994 (DSHEA) and is not intended to diagnose, treat, prevent, mitigate or cure any disease.

Without further elaboration, it is believed that one skilled in the art can, using the preceding description, utilize the exemplary embodiments described to their fullest extent. The specific formulas are included as exemplary embodiments of the composition formula ranges, and not to further qualify the description. It is preferred, therefore, that the exemplary embodiments are limited not by the specific disclosure herein, but only by the claims. Claim references to specific components include the component itself, as well as concentrates, metabolites, constituents, extracts or combinations of said ingredients.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components unless the context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations or two or more recitations). Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

1. A nutritional supplement, comprising:
a base formula, the base formula being formed from at least three categories of ingredients, the three categories being adaptogens, bio-enhancers, and circulation enhancers; and
a nutrient formula, the nutrient formula being formed of at least one ingredient having an intended targeted effect on a user,
wherein the adaptogens are combined from at least three adaptogenic ingredients,
wherein the bio-enhancers are combined from at least three bio-enhancing ingredients, and
wherein the circulation enhancers are combined from at least three circulation enhancing ingredients.

2. The nutritional supplement of claim 1, wherein the adaptogens are combined from at least five adaptogenic ingredients.

3. The nutritional supplement of claim 1, wherein the adaptogens are selected from the adaptogenic group consisting of:
at least one species of Eleuthero, at least one species of Ajuga turkestanica, at least one type of Shilajit, at least one type of Fulvic Acid, at least one type of Mumie, at least one species of Rhodiola, at least one species of Ginseng, at least one species of Rhaponticum, at least one type of Ecdysteroid, at least one species of Ashwagandha, at least one species of Holy Basil, at least one species of Aralia, at least one species of Prince Seng, at least one species of Dong Quai, at least one species of Maca, at least one species of Schisandra, at least one type of Cannabidiol, at least one species of He Shou Wu, at least one type of Turkesterone, at least one species of Amla, at least one species of Sorbus aucuparia, at least one species of Viburnum Sargentii, at least one species of Rosa majalis, at least one type of Alpha-Casozepine, at least one type of Ecdysterone, at least one species of Jiaogulan, at least one species of Gynostemma pentaphyllum, at least one species of Cyanotis, at least one species of chaga, at least one species of Cordyceps, at least one species of Lycium, at least one species of reishi, at least one type of Pantocrine, at least one type of Guggulsterone, at least one species of Astragalus, at least one species of suma, at least one species of Dang Shen, at least one species of Shatavari, at least one species of Guduchi, and at least one species of Asparagus racemosus.

4. The nutritional supplement of claim 1, wherein the adaptogens combined from at least five adaptogenic ingredients and the at least five adaptogens the adaptogens are selected from the adaptogenic group consisting of:
at least one species of Eleuthero, at least one species of Ajuga Turkestanica, at least one type of Shilajit, at least one type of Fulvic Acid, at least one type of Mumie, at least one species of Rhodiola, at least one species of Ginseng, at least one species of Rhaponticum, at least one type of Ecdysteroid, at least one species of Ashwagandha, at least one species of Holy Basil, at least one species of Aralia, at least one species of Prince Seng, at least one species of Dong Quai, at least one species of Maca, at least one species of Schisandra, at least one type of Cannabidiol, at least one species of He Shou Wu, at least one type of Turkesterone, at least one species of Amla, at least one species of Sorbus aucuparia, at least one species of Viburnum sargentii, at least one species of Rosa majalis, at least one type of Alpha-Casozepine, at least one type of Ecdysterone, at least one species of Jiaogulan, at least one species of Gynostemma pentaphyllum, at least one species of Cyanotis, at least one species of chaga, at least one species of Cordyceps, at least one species of Lycium, at least one species of reishi, at least one type of Pantocrine, at least one type of Guggulsterone, at least one species of Astragalus, at least one species of suma, at least one species of Dang Shen, at least one species of Shatavari, at least one species of Guduchi, and at least one species of Asparagus racemosus.

5. The nutritional supplement of claim 1, wherein the bio-enhancers are combined from at least five bio-enhancing ingredients.

6. The nutritional supplement of claim 1, wherein the bio-enhancers are selected from the bio-enhancing group consisting of:
at least one species of Ginger, at least one species of Licorice, at least one species of Pepper, at least one type of Quercetin, at least one species of Grapefruit, at least one type of Bergamotine, at least one type of Dihydroxybergamottin, at least one type of Fulvic Acid, and at least one type of Nanarin.

7. The nutritional supplement of claim 1, wherein the bio-enhancers are combined from at least five bio-enhancing ingredients selected from the bio-enhancing group consisting of:
at least one species of Ginger, at least one species of Licorice, at least one species of Pepper, at least one type of Quercetin, at least one species of Grapefruit, at least one type of Bergamotine, at least one type of Dihydroxybergamottin, at least one type of Fulvic Acid, and at least one type of Nanarin.

8. The nutritional supplement of claim 1, wherein the circulation enhancers are combined from at least three circulation enhancing ingredients.

9. The nutritional supplement of claim 1, wherein the circulation enhancers are selected from the circulation enhancing group consisting of:
at least one type of Vitamin D, at least one type of Niacin, at least one type of Magnesium, at least one type of Calcium, at least one type of Silica, at least one type of Vitamin C, at least one species of Spinach, at least one species of Hawthorn, at least one species of Pine Bark, at least one species of Artichoke, at least one species of Ginkgo, at least one species of Green Tea, at least one type of Vitamin B12, at least one type of Vitamin K2 (as menaquinone), at least one type of Arginine, at least one type of Citrulline, at least one species of Beetroot, at least one type of Nitrate, at least one type of Nitrite, at least one species of Rhubarb, at least one species of Arugula, at least one species of Butter Leaf Lettuce, and at least one species of Pepper.

10. The nutritional supplement of claim 1, wherein the circulation enhancers are combined from at least five circulation enhancing ingredients selected from the circulation enhancing group consisting of:
at least one type of Vitamin D, at least one type of Niacin, at least one type of Magnesium, at least one type of Calcium, at least one type of Silica, at least one type of Vitamin C, at least one species of Spinach, at least one species of Hawthorn, at least one species of Pine Bark, at least one species of Artichoke, at least one species of Ginkgo, at least one species of Green Tea, at least one type of Vitamin B12, at least one type of Vitamin K2 (as menaquinone), at least one type of Arginine, at least one type of Citrulline, at least one species of Beetroot, at least one type of Nitrate, at least one type of Nitrite, at least one species of Rhubarb, at least one species of Arugula, at least one species of Butter Leaf Lettuce, and at least one species of Pepper.

11. The nutritional supplement of claim 1, wherein the nutrient formula is combined from at least at least ten agents selected from the group consisting of:
at least one species of Coleus forskohlii, at least one species of Cissus quadrangularis, at least one type of Iodine, at least one type of Selenium, at least one type of Vitamin B12, at least one type of Vanadium, and at least one type of Manganese, at least one type of Chromium, at least one type of Boron, at least one species of Garcinia cambogia, at least one species of Eurycoma longifolia, at least one type of DHEA, at least one species of Tribulus terrestris, at least one type of L-dopa, at least one type of Vitamin B6, at least one species of Ashitaba, at least one type of Zinc, at least one type of Laxogenin, at least one species of Fenugreek, at least one type of Glucomannan, at least one type of Pregnenolone, at least one type of Corosolic Acid, at least one species of Irvingia gabonensis, at least one species of Shatavari, at least one species of Anacyclus pyrethrum, at least one species of White Button Mushroom, at least one species of Saw palmetto, at least one species of Green Tea, at least one species of Green Coffee Bean, at least one species of Citrus aurantium, at least one species of Gynostemma, at least one species of Dong Quai, at least one species of Lotus Leaf, at least one species of Oats, at least one type of D-aspartic acid, at least one species of Black Cohosh, at least one species of Chasteberry, at least one type of Secoisolariciresinol diglucoside, at least one type of Diindolylmethane, at least one type of 5-Hydroxytryptophan, and at least one type of Galanthamine.

12. The nutritional supplement of claim 1, wherein the nutrient formula is combined from at least at least ten agents selected from the group consisting of:
at least one type of Molybdenum, at least one type of Astaxanthin, at least one species of Huperzine A, at least one species of Garlic, at least one species of Artichoke, at least one type of Vitamin K2, at least one type of Vitamin C, at least one type of Vitamin E, at least one type of Coenzyme Q10, at least one type of Nicotinamide Riboside (NAD+), at least one type of Nicotinamide Mononucleotide (NMN), at least one type of Sodium Hydrosulfide, at least one species of Arjuna, at least one type of Lipoic Acid, at least one type of Folate, at least one type of Quercetin, at least one species of Guggul, at least one species of Green Tea, at least one type of Policosanol, at least one species of Cinnamon Bark, at least one species of Motherwort, at least one type of Chrysin, at least one species of Goat's Rue, at least one species of Green Coffee Bean, at least one species of Grape Seed, at least one species of Hawthorn, at least one type of Bromelain, at least one species of Linden Flower, at least one type of Curcumin, at least one type of Turmeric, at least one type of NADH (Nicotinamide Adenine Dinucleotide), at least one type of Bitter Melon, at least one type of Resveratrol, and at least one type of Nattokinase or Lumbrokinase.

13. The nutritional supplement of claim 1, wherein the nutrient formula is combined from at least at least ten agents selected from the group consisting of:
at least one species of Ginkgo biloba, at least one species of Gotu Kola, at least one type of Saffron, at least one species of Kanna, at least one species of Rosemary, at least one species of Lemon Balm, at least one type of Methylliberine, at least one species of Lion's Mane, at least one species of Bacopa monari, at least one species Maritime Pine Bark, at least one type of Phenylpiracetam, at least one species of Passionflower, at least one type of Folate, at least one species of Bilberry, at least one type of Resveratrol, at least one type Silk Protein Hydrolysate, at least one type of 7,8 Dihydroxyflavone, at least one type of Nicotinamide Riboside (NAD+), at least one type of Nicotinamide Mononucleotide (NMN), at least one type of Nicotinamide Adenine Dinucleotide (NADH), at least one species of Celastrus paniculatus, at least one type of Lithium, at least one type of PQQ Disodium Salt, at least one type of Uridine, at least one type of Centrophenoxine, at least one type of Tianeptine Sulfate, at least one species of Sage, at least one type of Vinpocetine, at least one type of Galantamine, at least one type of Phosphatidylserine (PS), at least one type of Nicotine, at least one type of Gamma-Aminobutyric Acid (GABA), at least one type of Fasoracetam, at least one type of Nattokinase, at least one type of Adrafinil, at least one type of Diindolylmethane (DIM), at least one type of Lipoic Acid, at least one type of Coenzyme Q10, at least one type of Huperzine A, at least one type of Hesperidin, at least one type of Curcumin, at least one type of Theacrine, at least one type of Coline, at least one type of Inositol, at least one type of Methionine, at least one type of L-Theanine, at least one type of L-Tyrosine, at least one type of Carnitine, at least one type of Phycocyanin, at least one type of DHA (Docosahexaenoic acid), at least one type of Boron, at least one type of Coluracetam, at least one type of Zinc, at least one type of Vitamin B6, at least one type of S-HTP, at least one type of Iodine, at least one type of Noopept, at least one type of Magnesium, and at least one type of L-Dopa.

14. A nutraceutical product, comprising:
at least three adaptogenic ingredients each potentially having adaptogenic effects;
at least three bio-enhancing ingredients each potentially having bio-enhancing effects;
at least three circulation enhancing ingredients each potentially having circulation enhancing effects; and
at least one nutrient ingredient, the nutrient ingredient potentially having an intended targeted effect on a user,
wherein the adaptogenic ingredients, the bio-enhancing ingredients, and the nutrient ingredients are combined in a form suitable for consumption by a human being.

15. The nutritional supplement of claim 14, wherein the nutrient ingredient is combined from at least at least three agents selected from the group consisting of:
at least one species of Coleus forskohlii, at least one species of Cissus quadrangularis, at least one type of Iodine, at least one type of Selenium, at least one type of Vitamin B12, at least one type of Vanadium, and at least one type of Manganese, at least one type of Chromium, at least one type of Boron, at least one species of Garcinia cambogia, at least one species of Eurycoma longifolia, at least one type of DHEA, at least one species of Tribulus terrestris, at least one type of L-dopa, at least one type of Vitamin B6, at least one species of Ashitaba, at least one type of Zinc, at least one type of Laxogenin, at least one species of Fenugreek, at least one type of Glucomannan, at least one type of Pregnenolone, at least one type of Corosolic Acid, at least one species of Irvingia gabonensis, at least one species of Shatavari, at least one species of Anacyclus pyrethrum, at least one species of White Button Mushroom, at least one species of Saw palmetto, at least one species of Green Tea, at least one species of Green Coffee Bean, at least one species of Citrus aurantium, at least one species of Gynostemma, at least one species of Dong Quai, at least one species of Lotus Leaf, at least one species of Oats, at least one type of D-aspartic acid, at least one species of Black Cohosh, at least one species of Chasteberry, at least one type of Secoisolariciresinol diglucoside, at least one type of Diindolylmethane, at least one type of 5-Hydroxytryptophan, and at least one type of Galanthamine.

16. The nutritional supplement of claim 14, wherein the nutrient formula is combined from at least at least three agents selected from the group consisting of:
at least one type of Molybdenum, at least one type of Astaxanthin, at least one species of Huperzine A, at least one species of Garlic, at least one species of Artichoke, at least one type of Vitamin K2, at least one type of Vitamin C, at least one type of Vitamin E, at least one type of Coenzyme Q10, at least one type of Nicotinamide Riboside (NAD+), at least one type of Nicotinamide Mononucleotide (NMN), at least one type of Sodium Hydrosulfide, at least one species of Arjuna, at least one type of Lipoic Acid, at least one type of Folate, at least one type of Quercetin, at least one species of Guggul, at least one species of Green Tea, at least one type of Policosanol, at least one species of Cinnamon Bark, at least one species of Motherwort, at least one type of Chrysin, at least one species of Goat's Rue, at least one species of Green Coffee Bean, at least one species of Grape Seed, at least one species of Hawthorn, at least one type of Bromelain, at least one species of Linden Flower, at least one type of Curcumin, at least one type of Turmeric, at least one type of NADH (Nicotinamide Adenine Dinucleotide), at least one type of Bitter Melon, at least one type of Resveratrol, and at least one type of Nattokinase or Lumbrokinase.

17. The nutritional supplement of claim 14, wherein the nutrient formula is combined from at least at least three agents selected from the group consisting of:
at least one species of Ginkgo biloba, at least one species of Gotu Kola, at least one type of Saffron, at least one species of Kanna, at least one species of Rosemary, at least one species of Lemon Balm, at least one type of Methylliberine, at least one species of Lion's Mane, at least one species of Bacopa monari, at least one species Maritime Pine Bark, at least one type of Phenylpiracetam, at least one species of Passionflower, at least one type of Folate, at least one species of Bilberry, at least one type of Resveratrol, at least one type Silk Protein Hydrolysate, at least one type of 7,8 Dihydroxyflavone, at least one type of Nicotinamide Riboside (NAD+), at least one type of Nicotinamide Mononucleotide (NMN), at least one type of Nicotinamide Adenine Dinucleotide (NADH), at least one species of Celastrus paniculatus, at least one type of Lithium, at least one type of PQQ Disodium Salt, at least one type of Uridine, at least one type of Centrophenoxine, at least one type of Tianeptine Sulfate, at least one species of Sage, at least one type of Vinpocetine, at least one type of Galantamine, at least one type of Phosphatidylserine (PS), at least one type of Nicotine, at least one type of Gamma-Aminobutyric Acid (GABA), at least one type of Fasoracetam, at least one type of Nattokinase, at least one type of Adrafinil, at least one type of Diindolylmethane (DIM), at least one type of Lipoic Acid, at least one type of Coenzyme Q10, at least one type of Huperzine A, at least one type of Hesperidin, at least one type of Curcumin, at least one type of Theacrine, at least one type of Coline, at least one type of Inositol, at least one type of Methionine, at least one type of L-Theanine, at least one type of L-Tyrosine, at least one type of Carnitine, at least one type of Phycocyanin, at least one type of DHA (Docosahexaenoic acid), at least one type of Boron, at least one type of Coluracetam, at least one type of Zinc, at least one type of Vitamin B6, at least one type of S-HTP, at least one type of Iodine, at least one type of Noopept, at least one type of Magnesium, and at least one type of L-Dopa.

18. A method of manufacturing a nutraceutical product, comprising:
combining into a mixture at least three adaptogenic ingredients each potentially having adaptogenic effects;
combining into the mixture at least three bio-enhancing ingredients each potentially having bio-enhancing effects;
combining into the mixture at least three circulation enhancing ingredients each potentially having circulation enhancing effects;
combining into the mixture at least one nutrient ingredient, the nutrient ingredient potentially having an intended targeted effect on a user; and
forming the mixture suitable for consumption by a human being.

19. The method of claim 18, wherein the mixture comprises a capsule, sublingual tablet, powder, liquid beverage, gel, or food bar.

20. The method of claim 18, wherein the neutraceutical product is manufactured in a cGMP compliant facility.
